(19) United States
(12) Patent Application Publication
(10) Pub. No.: US 2012/0029451 A1
(43) Pub. Date: Feb. 2, 2012

(54) MALE EXTERNAL URINARY INCONTINENCE DEVICE

(76) Inventor: Anthony J. Conway, Chatfield, MN (US)

(21) Appl. No.: 13/194,148

(22) Filed: Jul. 29, 2011

Related U.S. Application Data
(60) Provisional application No. 61/369,336, filed on Jul. 30, 2010.

(51) Int. Cl. A61F 5/453

(52) U.S. Cl. 604/349; 427/2.3

(57) ABSTRACT

The present invention relates to a male external urinary incontinence device including a hydrocolloid composition as an adhesive and to methods of making and using this device. The hydrocolloid composition can include an acrylic pressure sensitive adhesive, a polyacrylic acid polymer, and, optionally, a neutralizer.

### CROSS REFERENCE TO RELATED APPLICATION

This application claims the benefit of provisional application Ser. No. 61/369,336, filed Jul. 30, 2010, which application is incorporated herein by reference in its entirety.

### FIELD

The present invention relates to a male external urinary incontinence device including a hydrocolloid composition as an adhesive and to methods of making and using this device. The hydrocolloid composition can include an acrylic pressure sensitive adhesive, a polyacrylic acid polymer, and, optionally, a neutralizer.

### BACKGROUND

Male urinary incontinence devices of the type using a urine receptacle worn on or near the body can use a sleeve of flexible material placed over the penis and connected to the receptacle with a tube or other form of flexible conduit. Some devices customarily include a narrow tape wound relatively tightly about the sleeve with the dual intention to hold the sleeve securely and to form a seal to prevent leakage of urine. Since urinary incontinence devices must be worn for long periods of time, particularly in the case of paraplegics, wound tape can exert continuous pressure on a penis, particularly on the urethral passage which is located on the under side of the penis, fairly close to the surface. Constrictions, lesions and discomfort may result. Other constructions have used adhesive to adhere the catheter to the penis to avoid inadvertent removal and avoid leakage of urine. Catheter devices with adhesive can cause unacceptable maceration of the skin and irritation if frequent replacement is required.

### SUMMARY

The present invention relates to a male external urinary incontinence device including a hydrocolloid composition as an adhesive and to methods of making and using this device. The hydrocolloid composition can include an acrylic pressure sensitive adhesive, a polyacrylic acid polymer, and, optionally, a neutralizer.

The present invention includes a condom catheter including a hydrocolloid composition. In an embodiment, the condom catheter can include a tubular sleeve member of resilient material. The sleeve member can be in a configuration in which it is rolled outwardly upon itself. The sleeve member can have an outer surface and an inner surface. When in the rolled configuration, the outer surface of the sleeve member contacts a layer of hydrocolloid composition and the layer of hydrocolloid composition contacts the inner surface of the sleeve member.

In an embodiment, the condom catheter can include a silicone rubber sheath including an inner surface and an outer surface and a layer of hydrocolloid composition directly and non-releasably bonded to a first portion of the inner surface. In this embodiment, the hydrocolloid composition can releasably contact portions of the outer surface such that the inner surface of the silicone rubber sheath can be rolled up upon the outer surface. The layer of hydrocolloid composition on the first portion of the inner surface comes into releasable contact with portions of the outer surface. The hydrocolloid composition on the outer surface will release any portions of the outer surface with which it has come into contact, while remaining bonded to the inner surface, when the silicone rubber sheath is forcibly unrolled.

The present invention also includes a method of making a condom catheter. This method includes applying a hydrocolloid composition to a portion of a mandrel; applying silicone rubber to the mandrel and over the hydrocolloid composition; curing the silicone rubber to form a condom catheter comprising a layer of hydrocolloid composition; and removing the condom catheter from the mandrel.

The present invention also includes a method of catheterizing a subject. This method includes unrolling a condom catheter on a penis of the subject to bring a hydrocolloid composition into contact with the penis. The hydrocolloid composition adheres the condom catheter to the penis.

The present invention also includes a hydrocolloid adhesive composition. The hydrocolloid prep composition can include about 90 to about 99.99 wt-% of acrylic pressure sensitive adhesive and solvent (about 35 to about 45 wt-% solids (e.g., the adhesive composition) the remainder being solvent); about 0.01 to about 10 wt-% (e.g., about 0.5 wt-%) polyacrylic acid polymer; and, optionally, about 0.01 to about wt-% (e.g., about 0.5 wt-%) neutralizer. The neutralizer and polyacrylic acid polymer can be in a ratio of about 0.9 to about 1 (e.g., 0.9:1). Once dried or cured on the catheter or mandrel, the hydrocolloid adhesive composition can include about 55 to about 99.98 wt-% of acrylic pressure sensitive adhesive; about 0.02 to about 30 wt-% polyacrylic acid polymer; and, optionally, about 0.02 to about 15 wt-% neutralizer.

### BRIEF DESCRIPTION OF THE DRAWINGS

FIG. 1 is a cross-sectional view of a rolled-up condom catheter device in accordance with the present disclosure.

FIGS. 2-5 schematically illustrate a sequence showing use of an embodiment of the condom catheter of the present disclosure. FIG. 2 shows, in side view, contact of the cone with the glans. FIG. 3 shows, in side view, pulling the penis with the catheter device to expose the penile shaft from pelvic skin. FIG. 4 shows, in side view with a portion of the catheter cut away and adhesive remaining, how the unrolled sleeve fastens to the penile shaft. FIG. 5 shows, in side view with a portion of the sleeve cut away, how the penile shaft with sleeve fastened thereto recesses back into pelvic skin.

FIGS. 6-11 schematically illustrate a sequence showing a method of making a condom catheter device in accordance with the present disclosure. FIG. 6 shows a blank mandrel in side view. FIG. 7 shows a cross-sectional view of the mandrel having been dipped in hydrocolloid. FIG. 8 shows a cross-sectional view having been dipped in a solvent stripping agent. FIG. 9 shows a cross-sectional view having been dipped in silicone rubber. FIG. 10 shows a cross-sectional view having been dipped a subsequent time in silicon rubber to build the tube and lower cone region to a greater thickness. FIG. 11 shows in partial cross-sectional view a beginning roll of the sleeve as a part of removing a condom catheter from the mandrel.

FIG. 12 schematically illustrates an enlarged cross-sectional view of the sleeve portion rolled outwardly upon itself.

### DETAILED DESCRIPTION

The present invention relates to a male external urinary incontinence device (e.g., a condom catheter) including a hydrocolloid composition as an adhesive and to methods of making and using this device. The hydrocolloid composition can include an acrylic pressure sensitive adhesive, a polyacrylic acid polymer, and, optionally, a neutralizer. The polyacrylic acid polymer can be a cross-linked polyacrylic acid polymer. Such a crosslinked polymer can provide efficient rheology modification and enhanced self-wetting. Suitable polyacrylic acid polymers include that sold under the trade name CARBOPOL ULTREZ 10 NF®, by Lubrizol, Cleveland, Ohio. Suitable neutralizers include amines, such as an aminomethyl propanol. Suitable amino methyl propanols include that sold under the trade name AMP-95® by Angus, a subsidiary of Dow Chemical.

For making the catheter, the hydrocolloid prep composition can include about 90 to about 99.99 wt-% of acrylic pressure sensitive adhesive and solvent (about 35 to about 45 wt-% solids (e.g., the adhesive composition) the remainder being solvent); about 0.01 to about 10 wt-% (e.g., about 0.5 wt-%) polyacrylic acid polymer; and, optionally, about 0.01 to about 5 wt-% (e.g., about 0.5 wt-%) neutralizer. In an embodiment, the hydrocolloid prep composition can include about 90 to about 99.99 wt-% of acrylic pressure sensitive adhesive and solvent (about 35 to about 45 wt-% solids (e.g., the adhesive composition) the remainder being solvent); and about 0.01 to about 10 wt-% of polyacrylic acid polymer and neutralizer; the neutralizer and polyacrylic acid polymer being in a ratio of about 0.9 to about 1 (e.g., 0.9:1). The amount of neutralizer can be just sufficient to neutralize the polyacrylic acid polymer without adding additional basicity to a mixture of the polyacrylic acid and the neutralizer. In an embodiment, the neutralizer and polyacrylic acid polymer are in a ratio of 0.7-0.99:0.95-1.2 (e.g., 0.9:1). Once dried or cured on the catheter or mandrel, the hydrocolloid adhesive composition can include about 55 to about 99.98 wt-% of acrylic pressure sensitive adhesive; about 0.02 to about 30 wt-% polyacrylic acid polymer; and, optionally, about 0.02 to about 15 wt-% neutralizer.

The condom catheter can be of any of a variety of configurations with a tubular sleeve or a sheath that fits over a penis. The tubular sleeve or the sheath can be made of a resilient material (e.g., silicone rubber) and can have an inner surface and an outer surface. The hydrocolloid composition can be on a portion of the inner surface. In a package and before use, the tubular sleeve or the sheath can be rolled up upon itself. In the rolled up configuration, the tubular sleeve or sheath can isolate the hydrocolloid composition from the surroundings. For example, the hydrocolloid composition can be between layers of the rolled sleeve or sheath. In the rolled configuration, the outer surface of the sleeve or sheath can contact the layer of hydrocolloid composition and the layer of hydrocolloid composition can contact the inner surface of the sleeve or sheath. For example, the hydrocolloid composition can be between the inner and outer surfaces of one or more consecutive rolls so that the adhesive is covered by the inner surface when the sheath is rolled up.

In an embodiment, the hydrocolloid composition is in the form of a layer that is directly and non-releasably bonded to a portion of the inner surface. The hydrocolloid composition can releasably contact one or more portions of the outer surface. The inner surface of the silicone rubber sheath can be rolled up upon the outer surface. The layer of hydrocolloid composition on the portion of the inner surface comes into releasable contact with one or more portions of the outer surface. The hydrocolloid composition on the outer surface can then release from the outer surface with which it has come into contact, while remaining bonded to the inner surface, when the silicone rubber sheath is forcibly unrolled.

In an embodiment, the present male urinary incontinence device includes a condom for fitting about a normal, flaccid penis. The condom is coupled to a tube or catheter for carrying urine to a bag or other collection apparatus. The condom includes a flexibly cylindrical member rolled outwardly upon itself forming consecutively larger rolls. This cylindrical member has the hydrocolloid composition between consecutive rolls. The outer surface of the cylindrical member releases the hydrocolloid composition when the member is unrolled and inner surface of the cylindrical member retains the hydrocolloid when the member is unrolled. In this fashion, the device may be stored with the hydrocolloid composition protected between consecutive rolls of the member and used by unrolling the member onto a penis allowing the hydrocolloid to release from the outer surface and allowing the hydrocolloid to adhere to the inner surface and the penis.

In an embodiment, the male urinary incontinence device is in the form of an external male catheter having a sleeve or sleeve that can fit a normal, flaccid penis. The sleeve has an open end and an end opposite which unitarily joins a bulbous surge chamber and a catheter portion for connection to a tube leading to a collection receptacle. In a pre-operational position, the sleeve is outwardly rolled upon itself. The hydrocolloid composition is located between consecutive rolls. When the sleeve is rolled onto a penis, the hydrocolloid composition is between the sleeve and the penis adhering the sleeve to the penis. In this way, the hydrocolloid composition is sandwiched between the silicone rubber and the penis and upon applying an appropriate amount of pressure to the sleeve, hydrocolloid and penis forms a bond (e.g., a leak free bond).

The present invention also relates to a method of using a condom catheter. This method can include unrolling a condom catheter on a penis of the subject to bring a (e.g., the present) hydrocolloid composition into contact with the penis. The hydrocolloid composition adheres the condom catheter to the penis. In an embodiment, the method for installing the male external catheter onto a penis includes unrolling the sleeve or condom portion of the catheter onto the penis and pressing the condom portion against the penis to form around (e.g., completely around) the penis a bond between the condom portion and the penis with the aid of the hydrocolloid composition. The hydrocolloid composition is released from the outer surface of the condom portion and adheres to the inner surface of the condom portion during the unrolling step.

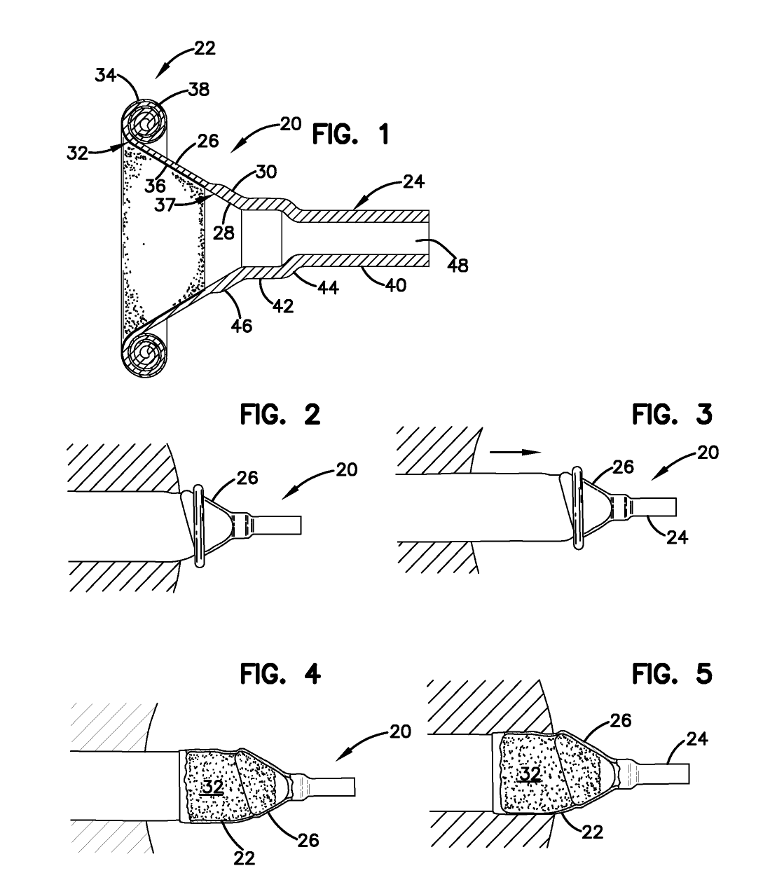

FIG. 1 schematically illustrates a male urinary incontinence device or condom catheter 20 according to certain embodiments of the present invention. Condom catheter 20 has a unitary construction which includes a sleeve 22, a tube 24, and a transition section 26 between the sleeve 22 and tube 24. Condom catheter 20 can be made of silicone rubber. The condom catheter 20 has an inner surface 28 and an outer surface 30. Following manufacture and during pre-use storage, condom catheter 20 has a pre-use configuration as shown in FIG. 1, wherein a first portion 34 of sleeve 22 is rolled up on itself so that except as indicated hereinafter inner surface 28 is rolled up against and comes into contact with outer surface 30. Interposed between most of inner surface 28 and outer surface 30 of sleeve 22 is a hydrocolloid layer 32. Hydrocolloid layer 32 adheres to inner surface 28 and does not adhere to outer surface 30 when sleeve 22 is unrolled. The reason for such phenomenon is discussed in more detail hereinafter. Hydrocolloid layer 32 also includes a second portion 36 formed on a significant portion of cone or transition section 26. First portion 34 and second portion 36 are contiguous as transition section 26 changes form to sleeve 22.

Sleeve 22 is formed as a cylinder having a diameter appropriate for a limp penis. Sleeve 22 has a length of approximately 1.2 inches. Such length can be long enough to provide sufficient fastening adhesion between first portion 34 of the hydrocolloid layer 32 on the inside surface of the sleeve and the penile shaft, but is not so long so that the sleeve cannot be completely unrolled when a recessed penis is pulled outwardly to expose the total length of penile shaft with respect to pelvic skin. First portion 34 includes all of the inner surface of sleeve 22 except a hydrocolloid-free band 38 on the inner surface of sleeve 22 adjacent its open end. Band 38 is intended to provide a loose end for a nurse to grasp and begin to roll sleeve 22 back on itself or otherwise to remove an installed condom catheter 20.

Cone or transition section 26 provides a reduction in diameter from sleeve 22 to tube 24. Second portion 36 to which hydrocolloid is applied contiguously with the hydrocolloid on first portion 34 includes most of the cone portion of transition section 26. In an embodiment, second portion 34 can provide an advantageously large hydrocolloid surface within cone 26 to contact and adhere to the glans penis. In an embodiment, an apex portion 37 which connects with and opens to tube 24 is free of hydrocolloid so that there is less chance that hydrocolloid can seal together in front of the glans penis when the catheter 20 is attached thereto. Only sleeve 22 is rolled in the pre-use configuration. Hence, second portion 36 on the inside surface 28 of transition section 26 is exposed (although the entire condom catheter 20 can be appropriately protected in a package). Sleeve 22 and transition section 26 associated with the second portion thereof have a thickness which allows them to be conformed to the shape of the penis as the hydrocolloid adheres to the penis. Tube 24 has a greater thickness so as to retain its shape and provide for suitable connection with additional tubing leading to a urine receptacle.

Tube 24 can be configured to provide a structure that retains its shape (e.g., doesn't collapse) when formed in a smaller diameter section 40 and a larger diameter section 42. A short transitional neck 44 extends between sections 40 and 42. Likewise, the narrow portion of the cone-shape of transition section 26 extends between larger diameter section 42 and the rest of transition section 26 so as to provide a short portion of greater thickness leading to the larger portion of transition section 26 having a thinner thickness. The distal end of tube 24 opens at opening 48 to provide for a urinary drainage tube junction designed to communicate through an appropriate drainage tube to a receptacle or collection device (not shown).

Although hydrocolloid layer 32 adheres to the inner surface 28, the hydrocolloid layer 32 does not adhere to the outer surface 30 when sleeve 22 is unrolled. Hydrocolloid layer 32 can be simply adhered to inner surface 28 or alternatively bonded to the inner surface 28 by a catalyzed process, for example, a vulcanizing process, in which constituents within the hydrocolloid composition are cross-linked to constituents within the silicone rubber which is formed from an unvulcanized silicone rubber solution overcoat layer during the vulcanizing process. Once the hydrocolloid layer 32 is adhered or bonded to the inner surface 28 and the outer surface is formed (e.g., according to a process described hereinafter), hydrocolloid 32 no longer irreversibly adheres to outer surface 30. Although the hydrocolloid will releasably adhere to outer surface 30, a moderate force separates the surfaces resulting in the hydrocolloid remaining adhered to the inner surface 28. Contact between hydrocolloid layer 32 and outer surface 30 is referred to as “releasable contact” or “releasable adherence.” As indicated, this type of contact or adherence is characterized in that it permits a relatively easy separation of the hydrocolloid from the contacting surface.

The present invention also includes a method of making the present condom catheter. This method can include applying a hydrocolloid composition to a portion of a mandrel; applying silicone rubber to the mandrel and over the hydrocolloid composition; curing the silicone rubber to form a condom catheter comprising a layer of hydrocolloid composition; and removing the condom catheter from the mandrel.

In certain embodiments, condom catheter 20 can be made by combining two or more layers of a silicone rubber solution or of separate silicone rubber solutions. Once the unvulcanized silicone rubber solutions are dried and cured in a vulcanizing process, the respective silicone rubber solution coatings are combined to form a single unitary wall without separate layers. Any silicone rubber solution used to form silicone rubber products can be used to form the silicone rubber catheter device of the present disclosure. The vulcanizing process can be a heat process, a catalyzed process employing a catalyzing agent or agents, a combination of the two, or any other suitable vulcanizing process known in the art.

The present condom catheter 20 can be employed on any of a variety of penis configurations, including a penis that is not recessed or one that is. The present condom catheter 20 can have a configuration advantageous for use with a recessed penis.

In an embodiment, condom catheter 20 functions both as a tool to position a recessed penis for installation and then functions as a condom catheter to direct urine from an incontinent male to an appropriate receptacle. The various steps of application and use are illustrated in FIGS. 2-5. A recessive penis has minimal exposure of the penile shaft from the pelvic area and may only expose the glans. Such minimal exposure has made it difficult at least, to fasten condom catheters in a safe and functional fashion. With respect to device 20, as illustrated in FIG. 2, the transition section 26 is positioned next to and brought into contact with the glans. The most flexible portion of the transition section is gently pressed and pinched against the glans so that the hydrocolloid adheres to the glans. In this embodiment, the installer then pulls with tube 24 by holding tube 24 between the two middle fingers of one hand. As the penis extends, the other hand is used to hold back the pelvic flesh. The thumb and index finger of the hand holding device 20 are used to unroll sleeve 22 onto the penile shaft. The sleeve is pressed all around so that the hydrocolloid adheres well and forms a good seal with the penis. The penis is extended sufficiently far from the pelvic skin so that the sleeve can be completely unrolled as indicated in FIG. 4. Alternatively, the installer may find it easier to pull the catheter 20 by holding the transition section 26, in adherence to the glans, between the thumb and forefinger of one hand, and to unroll the sleeve 22 with the thumb and forefinger of the other hand.

As indicated in FIG. 5, as the penis is allowed to recess back into the pelvic skin, the sleeve remains adjacent to the penile shaft and does not roll up and come off the penis, but rather remains in a functional configuration. It is clear that device 20 should be positioned so that urine is directed directly into tube 24 from the attachment of transition section 26 to the penile tip. When removal is desired, tube 24 is again used as a tool to pull the recessed penis with one hand while holding the pelvic skin back with the other. The second hand, in holding back the skin, can then also readily lift and peel the hydrocolloid-free band 38 of sleeve 22 from the penile shaft and either pull the entire sleeve back over itself or begin rolling it back over itself. In either case, the sleeve is gently released from adherence with the penile shaft and similarly the transition section is gently pulled from the glans for complete removal.

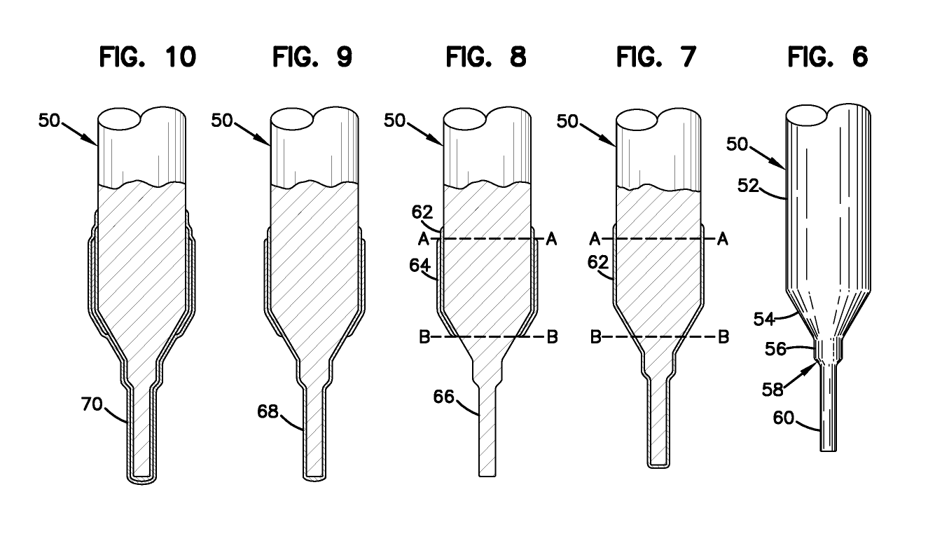

FIGS. 6-11 schematically illustrate an embodiment of a method of the present invention of making condom catheter 20. As shown in FIG. 6, mandrel 50 has a generally cylindrical shape which narrows from a large cylinder 52 along a major conical portion 54 to an intermediate cylindrical portion 56, from which it further narrows through a minor conical portion 58 to a narrow cylindrical portion 60. The surfaces of mandrel 50 can be coated with a material from which silicone rubber readily releases, such as a poly(tetrafluoroethane) (PTFE; available from E. I. du Pont de Nemours and Company under the trade designation “TEFLON®”). Mandrel 50 is first dipped into a tank containing hydrocolloid 62 so that hydrocolloid coats the mandrel at least up to line A (FIG. 7). Although many hydrocolloid compositions will adhere to unvulcanized silicone rubber a vulcanizing process can be used. The hydrocolloid composition can be the present hydrocolloid composition.

Mandrel 50 is next dipped in a solvent, for example, trichloroethane (trichlor 1, 1, 1) or xylene, which will strip the hydrocolloid coating 62 from the lower end 66 of mandrel 50 (FIG. 8). Mandrel 50 is dipped in the solvent only to line B so that the hydrocolloid 62 fully coats mandrel 50 between lines A and B. In this regard, it is noted that line B is relatively close to intermediate cylinder 56 so that hydrocolloid continues to cover a significant portion of the larger end of major conical portion 54.

After the cleaning step, as shown in FIG. 9, the mandrel is dipped in a tank containing a silicon rubber solution, for example, in siloxane or hexane as a solvent or in another solvent that does not destroy the integrity of the hydrocolloid strip which remains on the portion of the mandrel between lines A and B following the stripping step. When the mandrel 50 is dipped into the silicon rubber solution, the unvulcanized solution coats the mandrel 50 and overcoats the hydrocolloid 62 to form a first overcoat layer 68 which extends beyond the top end of hydrocolloid 62. The unvulcanized silicone rubber overcoat layer 68 is then allowed to dry.

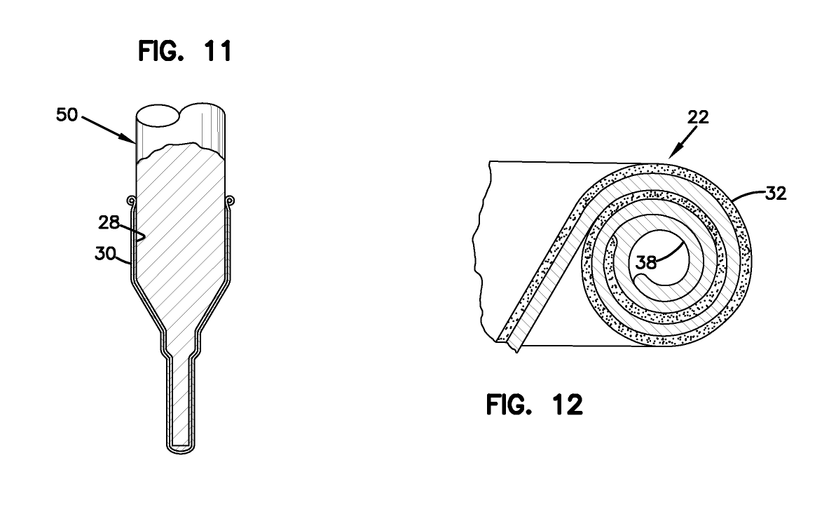

Additional layers of silicone rubber solution can be added to obtain the desired thickness. For example, optional silicone rubber layer 64 (FIG. 8) can be applied over the hydrocolloid layer and stripped from the lower portions of the mandrel. In an embodiment, an optional additional layer of silicone rubber can be applied to the lower portion of mandrel 50 to add greater thickness and structure to the tubular portion of condom catheter 20 up to approximately where the hydrocolloid remains. This additional thickness silicone rubber coating 70 is illustrated in FIG. 10.

After layer 70 is allowed to dry and any additional layers applied to obtain desired thickness, the final silicone rubber overcoat layer along the entire length of condom catheter 20 is vulcanized or cured at an elevated temperature, for example, about 205° F. The temperature can be maintained below the boiling point of the solvent or solvents used in the silicone rubber solutions. Other vulcanizing processes are applicable in some embodiments. During the vulcanizing process, any distinctions between the initial silicone rubber solution and any subsequent layers are eliminated and a single unitary silicone rubber wall throughout device 20 is formed.

Device 20 is allowed to cool and, as shown in FIG. 11, device 20 is then rolled from the top of mandrel 50 so that the inner surface 28 is rolled up onto outer surface 30. In the process of rolling, hydrocolloid layer 32 which has now been integrally bonded with the silicone rubber during the vulcanizing process, comes into contact with the outer surface 30 (FIG. 12). When the sleeve 22 is completely rolled, the conical portion and smaller tubular portions are pulled or otherwise removed from the mandrel and the end of the smallest tubular portion cut off to create opening 48.

The hydrocolloid is selected for its ability to bond with silicone rubber during the vulcanized process and for its lack of adherence when it comes into contact with vulcanized silicone rubber after the vulcanizing process. Furthermore, the hydrocolloid must adhere sufficiently to the penis, while at the same time release from it without undue discomfort. Particularly, it must release from the glans portion of the penis without injury or undue discomfort.

In other embodiments, the rolled external male catheter requires no additional liner to the hydrocolloid adhesive. The outer surface of the sheath that can be coated with, for example, a release layer, effectively can act as a “built-in” liner. Thus, some embodiments of the present application are easier to use in that there are fewer steps required and less waste than those requiring a liner to the hydrocolloid. Additionally, in some embodiments, the hydrocolloid layer can be thin thus allowing for less bulky, more comfortable fit to the wearer.

### Hydrocolloids

The condom catheter can include a hydrocolloid layer as described above. The hydrocolloid can have (strong) adhesive properties and compatibility with both skin and catheter materials. A suitable hydrocolloid composition includes an acrylic pressure sensitive adhesive, a polyacrylic acid polymer, and, optionally, a neutralizer. Such a hydrocolloid composition can be referred to as a “hydrocolloid adhesive composition”. The polyacrylic acid polymer can be a cross-linked polyacrylic acid polymer. Such a crosslinked polymer can provide efficient rheology modification and enhanced self-wetting. Suitable polyacrylic acid polymers include that sold under the trade name CARBOPOL ULTREZ 10 NF® by Lubrizol, Cleveland, Ohio. Suitable neutralizers include amines, such as an aminomethyl propanol, e.g., 2-amino-2-methyl-1-propanol.

Suitable amino methyl propanols (e.g., 2-amino-2-methyl-1-propanol) include that sold under the trade name AMP-95® by Angus, a subsidiary of Dow Chemical. The commercial product includes 93-97 wt-% 2-amino-2-methyl-1-propanol and about 5% water. In certain embodiments, the amounts of neutralizer listed in the present application can be multiplied by, for example, about 0.93, about 0.97, or about 0.93 to about 0.97 to account for the level of active in the commercial product. In certain embodiments, the amount of neutralizer listed in the present application refers to a neutralizer stock solution that includes about 93-97 wt-% 2-amino-2-methyl-1-propanol.

For making the catheter, the hydrocolloid prep composition can include about 90 to about 99.99 wt-% of acrylic pressure sensitive adhesive and solvent (about 35 to about 45 wt-% solids (e.g., the adhesive composition) the remainder being solvent); about 0.01 to about 10 wt-% (e.g., about 0.5 wt-%) polyacrylic acid polymer; and, optionally, about 0.01 to about 5 wt-% (e.g., about 0.5 wt-%) neutralizer. In an embodiment, the hydrocolloid prep composition can include about 90 to about 99.99 wt-% of acrylic pressure sensitive adhesive and solvent (about 35 to about 45 wt-% solids (e.g., the adhesive composition) the remainder being solvent); about 0.01 to about 10 wt-% (e.g., about 0.5 wt-%) polyacrylic acid polymer; and about 0.01 to about 5 wt-% (e.g., about 0.5 wt-%) neutralizer. In an embodiment, the hydrocolloid prep composition can include about 96 to about 99.6 wt-% of acrylic pressure sensitive adhesive and solvent (about 35 to about 45 wt-% solids (e.g., the adhesive composition) the remainder being solvent); about 0.2 to about 2 wt-% polyacrylic acid polymer; and about 0.2 to about 2 wt-% neutralizer. In an embodiment, the hydrocolloid prep composition can include about 98 to about 99.4 wt-% of acrylic pressure sensitive adhesive and solvent (about 35 to about 45 wt-% solids (e.g., the adhesive composition) the remainder being solvent); about 0.3 to about 1 wt-% polyacrylic acid polymer; and about 0.3 to about 1 wt-% neutralizer. In an embodiment, the hydrocolloid prep composition can include about 99 wt-% of acrylic pressure sensitive adhesive and solvent (about 35 to about 45 wt-% solids (e.g., the adhesive composition) the remainder being solvent); about 0.5 wt-% polyacrylic acid polymer; and about 0.5 wt-% neutralizer.

In an embodiment, the hydrocolloid prep composition can include about 90 to about 99.99 wt-% of acrylic pressure sensitive adhesive and solvent (about 35 to about 45 wt-% solids (e.g., the adhesive composition) the remainder being solvent). In an embodiment, the hydrocolloid prep composition can include about 96 to about 99.6 wt-% of acrylic pressure sensitive adhesive and solvent (about 35 to about 45 wt-% solids (e.g., the adhesive composition) the remainder being solvent). In an embodiment, the hydrocolloid prep composition can include about 98 to about 99.4 wt-% of acrylic pressure sensitive adhesive and solvent (about 35 to about 45 wt-% solids (e.g., the adhesive composition) the remainder being solvent). In an embodiment, the hydrocolloid prep composition can include about 99 wt-% of acrylic pressure sensitive adhesive and solvent (about 35 to about 45 wt-% solids (e.g., the adhesive composition) the remainder being solvent).

In an embodiment, the hydrocolloid prep composition can include about 0.01 to about 10 wt-% (e.g., about 0.5 wt-%) polyacrylic acid polymer. In an embodiment, the hydrocolloid prep composition can include about 0.2 to about 2 wt-% polyacrylic acid polymer. In an embodiment, the hydrocolloid prep composition can include about 0.3 to about 1 wt-% polyacrylic acid polymer. In an embodiment, the hydrocolloid prep composition can include about 0.5 wt-% polyacrylic acid polymer.

In an embodiment, the hydrocolloid prep composition includes no added neutralizer. In an embodiment, the hydrocolloid prep composition includes no neutralizer. In an embodiment, the hydrocolloid prep composition can include about 0.01 to about 5 wt-% (e.g., about 0.5 wt-%) neutralizer. In an embodiment, the hydrocolloid prep composition can include about 0.2 to about 2 wt-% neutralizer. In an embodiment, the hydrocolloid prep composition can include about 0.3 to about 1 wt-% neutralizer. In an embodiment, the hydrocolloid prep composition can include about 0.5 wt-% neutralizer.

Once dried or cured on the catheter or mandrel, the hydrocolloid adhesive composition can include about 55 to about 99.98 wt-% of acrylic pressure sensitive adhesive; about 0.02 to about 30 wt-% polyacrylic acid polymer; and, optionally, about 0.02 to about 15 wt-% neutralizer. In an embodiment, the hydrocolloid adhesive composition can include about 55 to about 99.96 wt-% of acrylic pressure sensitive adhesive; about 0.02 to about 30 wt-% polyacrylic acid polymer; and about 0.02 to about 15 wt-% neutralizer. In an embodiment, the hydrocolloid composition can include about 90 to about 99.4 wt-% of acrylic pressure sensitive adhesive; about 0.3 to about 5 wt-% polyacrylic acid polymer; and about 0.3 to about 5 wt-% neutralizer. In an embodiment, the hydrocolloid composition can include about 94 to about 99 wt-% acrylic pressure sensitive adhesive; about 0.5 to about 3 wt-% polyacrylic acid polymer; and about 0.5 to about 3 wt-% neutralizer. In an embodiment, the hydrocolloid composition can include about 97 to about 98 wt-% of acrylic pressure sensitive adhesive; about 1 to about 1.5 wt-% polyacrylic acid polymer; and about 1 to about 1.5 wt-% neutralizer. In an embodiment, the hydrocolloid composition can include about 97 wt-% of acrylic pressure sensitive adhesive; about 1.5 wt-% polyacrylic acid polymer; and about 1.5 wt-% neutralizer. In an embodiment, the hydrocolloid composition can include about 98 wt-% of acrylic pressure sensitive adhesive; about 1 wt-% polyacrylic acid polymer; and about 1 wt-% neutralizer.

Once dried or cured on the catheter or mandrel, the hydrocolloid adhesive composition can include about 55 to about 99.98 wt-% of acrylic pressure sensitive adhesive. In an embodiment, the hydrocolloid adhesive composition can include about 55 to about 99.96 wt-% of acrylic pressure sensitive adhesive. In an embodiment, the hydrocolloid composition can include about 90 to about 99.4 wt-% of acrylic pressure sensitive adhesive. In an embodiment, the hydrocolloid composition can include about 94 to about 99 wt-% acrylic pressure sensitive adhesive. In an embodiment, the hydrocolloid composition can include about 97 to about 98 wt-% of acrylic pressure sensitive adhesive. In an embodiment, the hydrocolloid composition can include about 97 wt-% of acrylic pressure sensitive adhesive. In an embodiment, the hydrocolloid composition can include about 98 wt-% of acrylic pressure sensitive adhesive.

Once dried or cured on the catheter or mandrel, the hydrocolloid adhesive composition can include about 0.02 to about 30 wt-% polyacrylic acid polymer. In an embodiment, the hydrocolloid composition can include about 0.02 to about 30 wt-% polyacrylic acid polymer. In an embodiment, the hydrocolloid composition can include about 0.3 to about 5 wt-% polyacrylic acid polymer. In an embodiment, the hydrocolloid composition can include about 0.5 to about 3 wt-% polyacrylic acid polymer. In an embodiment, the hydrocolloid composition can include about 1 to about 1.5 wt-% polyacrylic acid polymer. In an embodiment, the hydrocolloid composition can include about 1.5 wt-% polyacrylic acid polymer. In an embodiment, the hydrocolloid composition can include about 1 wt-% polyacrylic acid polymer.

Once dried or cured on the catheter or mandrel, the hydrocolloid adhesive composition can include about 0.02 to about 15 wt-% neutralizer. In an embodiment, the hydrocolloid composition can include about 0.02 to about 15 wt-% neutralizer. In an embodiment, the hydrocolloid composition can include about 0.3 to about 5 wt-% neutralizer. In an embodiment, the hydrocolloid composition can include about 0.5 to about 3 wt-% neutralizer. In an embodiment, the hydrocolloid composition can include about 1 to about 1.5 wt-% neutralizer. In an embodiment, the hydrocolloid composition can include about 1.5 wt-% neutralizer. In an embodiment, the hydrocolloid composition can include about 1 wt-% neutralizer.

The hydrocolloid can include a bioadhesive. Examples of suitable bioadhesives include poly(isobutylene) and acrylic adhesives. The hydrocolloid can be intimately mixed with the bioadhesive to produce an integral material.

Other suitable hydrocolloids include natural gums, such as plant exudates (gum arabic, ghatti, karaya, and tragacanth); plant seed gums (guar, locust bean and acacia), seaweed extracts (agar, algin, alginate salts and carrageenin), cereal gums (starches and modified starches), fermentation or microbial gums (dextran and xanthan gum), modified celluloses (hydroxymethylcellulose, microcrystalline cellulose and carboxymethylcellulose) pectin, gelatin, casein and synthetic gums (polyvinylpyrrolidone, low methoxyl pectin, propylene glycol alginates, carboxymethyl locust bean gum and carboxymethyl guar gum) and water-swellable or hydratable hydrocolloids. The term hydrocolloid is used regardless of the state of hydration. Hydrocolloid compositions can include a variety of components. A hydrocolloid composition can contain, for example, an adhesive base, a gelling agent, an absorptive agent, a setting agent, an anti-microbial agent, or a mixture thereof.

Suitable adhesive bases include polyisobutylenes and acrylics, both of which possess the desirable characteristics of biocompatibility and strong adhesiveness for skin and catheter materials. Other suitable adhesive bases include any of a variety of non-toxic polymers, for example, styrene, butadiene, styrene isoprene block copolymers, urethanes, silicones, styrene butadiene copolymers, methyl acrylate copolymers, acrylic acid, polyacrylates, and blends or copolymers thereof.

Suitable bases include bioadhesives for application to the epithelium that can be compatible with mucosal surfaces and exposed dermis. When a polyisobutylene (PIB) is used, a suitable type is a hot-melt, solvent-free compound with a high molecular weight (“MW”). An example of an acceptable high MW polyisobutylene is “VISTANEX® L-140” available from Exxon Corp. and having a MW in the range of from 117,000 to 135,000 daltons. The composition can include a low MW polyisobutylene, such as “VISTANEX® LMH”, with a MW in the range of from 11,600 to 12,300 daltons. The composition can include a high MW acrylic adhesive, such as “HRJ-4326”, with a MW in the range of 105,000 to 125,00 daltons, available from Schenectedy International, Inc., of Schenectedy N.Y. The composition can include a lower MW acrylic adhesive, such as “HRJ-10753”, with a MW in the range of 81,000-91,000 daltons, also available from Schenectedy International, Inc. In an embodiment of the hydrocolloid composition, the adhesive base makes up about 20 to about 60 wt-% of the hydrocolloid composition.

The term “bioadhesive” as used herein means an adhesive that adheres to or, for example, can strongly attach to a biological surface such as skin or mucosal tissue. Suitable bioadhesives include those that can maintain adhesion in moist or wet in vivo or in vitro environments. The strength of adherence of a hydrocolloid composition to a surface can be measured by standard tests for measuring the force, e.g. in dynes per square centimeter, as disclosed in U.S. Pat. No. 4,615,697.

Suitable gel agents include biocompatible compounds which, when exposed to water or aqueous solutions, form a solid gel, gelatin or highly viscous liquid. Suitable gel agents include sodium alginate, pectin, gelatin and agar.

Suitable absorptive agents include calcium silicate, and natural or synthetic polysaccharides. Suitable polysaccharides include cellulose derivatives such as methylcellulose, cellulose acetate, carboxymethylcellulose, hydroxyethylcellulose and the like.

Certain absorptive agents possess the capability to absorb and hold water or aqueous fluids in a colloidal mass. In a typical hydrated colloidal mass, the absorbed water or aqueous fluid may weigh many times the weight of the absorptive agent. These absorptive agents include polysacchrides such as, karaya gum, tragacanth gum, pectin, guar gum, cellulose, and cellulose derivatives such as methyl cellulose, propyl cellulose, cellulose acetate and the like, along with other substances known for use in forming a solid colloid that can adhere to skin and mucosa, used alone or in combination with various adhesive bases.

Other suitable absorptive agents include those prepared optionally from partially esterified polyacrylic acid polymers, including but not limited to, polyacrylic acid polymers lightly crosslinked with a polyalkenyl polyether such as those commercially available from B. F. Goodrich, Cincinnati, Ohio, under the trademarks “CARBOPOL® 934”, “CARBOPOL® 934P”, “CARBOPOL® 940” and “CARBOPOL® 941.”

Additional suitable absorptive agents include hydrophilic polysaccharide gums such as natural plant exudates, including karaya gum, ghatti gum, tragacanth gum, xanthan gum, jaraya gum and the like, as well as seed gums such as guar gum, locust bean gum, psillium seed gum and the like.

Setting agents suitable for the compositions include calcium salts such as calcium chloride, calcium phosphate and calcium sulphate. The corresponding magnesium salts may also be useful as setting agents.

Anti-microbial agents that can be employed in the hydrocolloid composition include an anti-fungal agent (e.g., magnesium borate) and other known topical anti-microbial agents, including bacitracin zinc, povidone iodine, benzalkonium chloride, neomycin sulfate, polymyxin B sulfate, silver sulfadiazine and mupirocin.

### Hydrocolloid Containing Silicone Sheath

In an embodiment, the present invention can include a sheath including a hydrocolloid silicone composition. A hydrocolloid silicone composition can include silicone rubber, a polyacrylic acid polymer, and, optionally, a neutralizer. In such an embodiment, the hydrocolloid composition can be a component of the adhesive layer, of one or more silicone rubber layers, or of both adhesive and silicone rubber layers.

In an embodiment, the present condom catheter can include a sleeve or sheath made of a hydrocolloid silicone composition. The present condom catheter can include a sleeve or sheath including a layer of hydrocolloid silicone composition disposed under the adhesive. Although not limiting to the present invention, it is believed that the hydrocolloid silicone composition beneath the adhesive can increase user comfort, for example, when removing the condom catheter. For example, the hydrocolloid silicone composition beneath the adhesive can reduce maceration of tissue (e.g., skin) to which the adhesive adheres.

In an embodiment, at least a portion of the resilient material can include a hydrocolloid. Or, both the adhesive and at least a portion of the resilient material include a hydrocolloid.

In an embodiment, the tubular sleeve or the sheath can be made of a resilient material (e.g., silicone rubber, a hydrocolloid silicone composition, or layers of both) and can have an inner surface and an outer surface.

In an embodiment of the present method of making the present condom catheter, the method can include applying a hydrocolloid silicone composition to the mandrel and over an adhesive composition; and curing the silicone rubber to form a condom catheter comprising a layer of hydrocolloid adhesive composition, a hydrocolloid silicone composition, or both; and removing the condom catheter from the mandrel.

Referring to the Figures, in an embodiment, condom catheter 20 can be made of silicone rubber (e.g., the present hydrocolloid silicone composition). In certain embodiments, condom catheter 20 can be made by combining two or more layers of a silicone rubber solution or of separate silicone rubber solutions. For example, one solution can be a silicone rubber solution and another solution can be a solution of a hydrocolloid silicone composition.

In an embodiment, after the cleaning step, as shown in FIG. 9, the mandrel is dipped in a tank containing a hydrocolloid silicone prep composition, for example, in siloxane or hexane as a solvent or in another solvent that does not destroy the integrity of the adhesive strip (e.g., hydrocolloid adhesive strip) which remains on the portion of the mandrel between lines A and B following the stripping step.

In an embodiment, when the mandrel 50 is dipped into the hydrocolloid silicone prep composition, the unvulcanized solution coats the mandrel 50 and overcoats the adhesive (e.g., hydrocolloid adhesive) 62 to form a first overcoat layer 68. The first overcoat layer 68 can extend beyond the top end of adhesive (e.g., hydrocolloid adhesive) 62. In an embodiment, the first overcoat layer is stripped so that is remains only on the adhesive (e.g., hydrocolloid adhesive) 62 or extending only a short distance beyond one or both ends of the adhesive (e.g., hydrocolloid adhesive) 62. The unvulcanized silicone rubber overcoat layer 68 is then allowed to dry. In an embodiment, optional silicone rubber layer 64 is made of hydrocolloid silicone composition.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

What is claimed is:

1. A condom catheter comprising:
a tubular sleeve member of resilient material rolled outwardly upon itself, the sleeve member having an outer surface and an inner surface;
wherein the outer surface of the sleeve member contacts a layer of hydrocolloid composition and the layer of hydrocolloid composition contacts the inner surface of the sleeve member.

2. The condom catheter of claim 1, wherein the tubular sleeve member has an integral conical portion of thicker material configured to connect to a urine collection device.

3. The condom catheter of claim 1, in which the hydrocolloid composition is between the inner and outer surfaces of one or more consecutive rolls so that the adhesive is covered by the inner surface when the sheath is in rolled up condition.

4. The condom catheter of claim 1, wherein the hydrocolloid composition comprises an acrylic pressure sensitive adhesive, a polyacrylic acid, and a polyethoxylated amine.

5. The condom catheter of claim 4, wherein the hydrocolloid composition comprises:
55 to about 99.98 wt-% of acrylic pressure sensitive adhesive;
about 0.02 to about 30 wt-% polyacrylic acid polymer; and,
optionally, about 0.02 to about 15 wt-% neutralizer.

6. A condom catheter, comprising:
a silicone rubber sheath comprising an inner surface and an outer surface; and
a layer of hydrocolloid composition directly and non-releasably bonded to a first portion of the inner surface;
wherein the hydrocolloid composition can releasably contact portions of the outer surface such that the inner surface of the silicone rubber sheath can be rolled up upon the outer surface thereof so that the layer of hydrocolloid composition on the first portion of the inner surface comes into releasable contact with portions of the outer surface, and such that the hydrocolloid composition on the outer surface will then release any portions of the outer surface with which it has come into contact, while remaining bonded to the inner surface, when the silicone rubber sheath is forcibly unrolled.

7. The condom catheter of claim 6, wherein further comprising an integral conical portion of thicker material configured to connect to a urine collection device.

8. The condom catheter of claim 6, wherein, when the catheter is rolled up, the hydrocolloid is between the inner and outer surfaces of one or more consecutive rolls so that the hydrocolloid is covered by the inner surface.

9. The condom catheter of claim 8, wherein the layer of hydrocolloid composition extends over at least one turn of the roll.

10. The condom catheter of claim 6, wherein the hydrocolloid composition comprises an acrylic pressure sensitive adhesive, a polyacrylic acid, and a polyethoxylated amine.

11. The condom catheter of claim 10, wherein the hydrocolloid composition comprises:
55 to about 99.98 wt-% of acrylic pressure sensitive adhesive;
about 0.02 to about 30 wt-% polyacrylic acid polymer; and,
optionally, about 0.02 to about 15 wt-% neutralizer.

12. A method of making a condom catheter, comprising:
applying a hydrocolloid composition to a portion of a mandrel;
applying silicone rubber to the mandrel and over the hydrocolloid composition;
curing the silicone rubber to form a condom catheter comprising a layer of hydrocolloid composition;
removing the condom catheter from the mandrel.

13. The method of claim 12, wherein the hydrocolloid composition comprises an acrylic pressure sensitive adhesive, a polyacrylic acid, and a polyethoxylated amine.

14. The method of claim 13, wherein the hydrocolloid composition comprises:
about 90 to about 99.99 wt-% of acrylic pressure sensitive adhesive and solvent
about 0.01 to about 10 wt-% polyacrylic acid polymer; and,
optionally, about 0.01 to about 5 wt-% neutralizer.

15. The method of claim 14, wherein the acrylic pressure sensitive adhesive and solvent comprises about 35 to about 45 wt-% solids.

16. A method of catheterizing a subject, comprising:
unrolling a condom catheter on a penis of the subject to bring a hydrocolloid composition into contact with the penis;
wherein the hydrocolloid composition adheres the condom catheter to the penis.

17. The method of claim 16, wherein the hydrocolloid comprises an acrylic pressure sensitive adhesive, a polyacrylic acid, and a polyethoxylated amine.

18. The method of claim 17, wherein the hydrocolloid composition comprises:
55 to about 99.98 wt-% of acrylic pressure sensitive adhesive;
about 0.02 to about 30 wt-% polyacrylic acid polymer; and,
optionally, about 0.02 to about 15 wt-% neutralizer.

19. An adhesive composition comprising:
about 90 to about 99.99 wt-% of acrylic pressure sensitive adhesive and solvent
about 0.01 to about 10 wt-% polyacrylic acid polymer; and,
optionally, about 0.01 to about 5 wt-% neutralizer.

20. The composition of claim 19, wherein the acrylic pressure sensitive adhesive and solvent comprises about 35 to about 45 wt-% solids.

21. An adhesive composition comprising:
55 to about 99.98 wt-% of acrylic pressure sensitive adhesive;
about 0.02 to about 30 wt-% polyacrylic acid polymer; and,
optionally, about 0.02 to about 15 wt-% neutralizer.

22. A condom catheter comprising:
a tubular sleeve member of resilient material rolled outwardly upon itself, the sleeve member having an outer surface and an inner surface;
wherein:
the outer surface of the sleeve member contacts a layer of adhesive composition and the layer of adhesive composition contacts the inner surface of the sleeve member; and
at least a portion of the tubular sleeve member comprises a hydrocolloid silicone composition.

23. The condom catheter of claim 22, wherein the hydrocolloid silicone composition comprises silicone rubber, a polyacrylic acid, and, optionally a neutralizer.

24. A method of making a condom catheter, comprising:
applying an adhesive composition to a portion of a mandrel;
applying silicone rubber to the mandrel and over the hydrocolloid composition;
curing the silicone rubber to form a condom catheter comprising a layer of hydrocolloid composition;
removing the condom catheter from the mandrel;
wherein at least a portion of the silicone rubber comprises a hydrocolloid silicone composition.

25. The condom catheter of claim 24, wherein the hydrocolloid silicone composition comprises silicone rubber, a polyacrylic acid, and, optionally a neutralizer.
